(19) United States
(12) Patent Application Publication
(10) Pub. No.: US 2023/0068745 A1
(43) Pub. Date: Mar. 2, 2023

(54) REAL-TIME REGISTRATION USING NEAR INFRARED FLUORESCENCE IMAGING

(71) Applicant: Covidien LP, Mansfield, MA (US)

(72) Inventor: Anthony B. Ross, Boulder, CO (US)

(21) Appl. No.: 17/464,955

(22) Filed: Sep. 2, 2021

(51) Int. Cl. A61B 5/00; A61B 1/012; A61B 1/04; A61B 5/06; A61B 1/00; A61K 49/00

(52) U.S. Cl. CPC A61B 5/0086; A61B 5/0071; A61B 5/0035; A61B 1/0125; A61B 1/04; A61B 5/062; A61B 1/0005; A61K 49/0034

(57) ABSTRACT

A system for performing a surgical procedure includes a camera configured to capture real-time near infrared images, an injection system configured to inject a fluorescent dye into a patient's blood stream, and a workstation operably coupled to the camera for retrieving a three-dimensional (3D) model of the patient's anatomy based on pre-procedure images, retrieve an indication of a targeted critical structure within the 3D model, observe, using the captured real-time near infrared images, perfusion of the fluorescent dye through tissue to identify critical structures illuminated by near-infrared light, and register the real-time near-infrared images to the 3D model using the identified illuminated targeted critical structure in the real-time near infrared images captured by the camera and the identified targeted critical structure in the 3D model as a landmark.

### BACKGROUND

### Technical Field

The present disclosure relates to the field of visualizing the navigation of medical devices, such as biopsy or ablation tools, relative to targets and confirming the relative positions with respect to generated three-dimensional models of patient anatomy.

### Description of Related Art

There are several commonly applied medical methods, such as endoscopic procedures or minimally invasive procedures, for treating various maladies affecting organs including the liver, brain, heart, lungs, gall bladder, kidneys, and bones. Often, one or more imaging modalities, such as magnetic resonance imaging (MRI), ultrasound imaging, computed tomography (CT), or fluoroscopy are employed by clinicians to identify and navigate to areas of interest within a patient and ultimately a target for biopsy or treatment. In some procedures, pre-operative scans may be utilized for target identification and intraoperative guidance. However, real-time imaging may be required to obtain a more accurate and current image of the target area. Furthermore, real-time image data displaying the current location of a medical device with respect to the target and its surroundings may be needed to navigate the medical device to the target in a safe and accurate manner (e.g., without causing damage to other organs or tissue).

For example, an endoscopic approach has proven useful in navigating to areas of interest within a patient. To enable the endoscopic approach endoscopic navigation systems have been developed that use previously acquired MRI data or CT image data to generate a three-dimensional (3D) rendering, model, or volume of the particular body part such as the lungs.

The resulting volume generated from the MM scan or CT scan is then utilized to create a navigation plan to facilitate the advancement of the endoscope (or other suitable medical device) within the patient anatomy to an area of interest. A locating or tracking system, such as an electromagnetic (EM) tracking system, may be utilized in conjunction with, for example, CT data, to facilitate guidance of the endoscope to the area of interest.

However, a 3D volume of a patient's lungs, generated from previously acquired scans, such as CT scans, may not provide a basis sufficient for accurate guiding of medical devices or instruments to a target during a navigation procedure. In some cases, the inaccuracy is caused by deformation of the patient's lungs during the procedure relative to the lungs at the time of the acquisition of the previously acquired CT data. This deformation (CT-to-Body divergence) may be caused by many different factors including, for example, changes in the body when transitioning from between a sedated state and a non-sedated state, the endoscope changing the patient's pose, the endoscope pushing the tissue, different lung volumes (e.g., the CT scans are acquired during inhale while navigation is performed during breathing), different beds, different days, etc.

While accurate, the accuracy of EM navigation systems can be improved to ensure a biopsy is taken from the correct tissue, especially instances where the lesion is small, and to ensure the proper margin is realized where target tissue must be resected. Thus, another modality in which the location of an endoscope can be registered to a generated 3D model is needed to enhance the accuracy of treating target tissue during endoscopic procedures.

### SUMMARY

In accordance with the present disclosure, a system for performing a surgical procedure includes a camera configured to capture real-time near infrared images, an injection system configured to inject a fluorescent dye into a patient's blood stream, and a workstation operably coupled to the camera, the workstation including a memory and a processor, the memory storing instructions, which when executed by the processor cause the processor to retrieve a three-dimensional (3D) model of the patient's anatomy based on pre-procedure images stored on the memory, receive an indication of a targeted critical structure within the 3D model, observe, using the captured real-time near infrared images, perfusion of the fluorescent dye through tissue to identify critical structures illuminated by near-infrared light, and register the real-time near infrared images to the 3D model using the identified illuminated targeted critical structure in the real-time near infrared images captured by the camera and the identified targeted critical structure in the 3D model as a landmark.

In aspects, the system may include a second camera configured to capture real-time white light images.

In other aspects, the system may include an endoscope, wherein the camera is disposed within a portion of the endoscope.

In certain aspects, the system may include an endoscope, wherein the camera and the second camera are disposed within a portion of the endoscope.

In other aspects, the fluorescent dye may be Indocyanine green dye.

In aspects, the instructions, when executed by the processor, may cause the processor to identify and segment a targeted critical structure from the generated 3D model.

In certain aspects, the system may include an electromagnetic (EM) sensor disposed within a portion of the endoscope, wherein the workstation is configured to sense the location of the EM sensor within the patient's body cavity.

In other aspects, the instructions, when executed by the processor, may cause the processor to register a location of the endoscope within the body cavity of the patient to the 3D model of the patient's anatomy using the captured real-time near infrared images.

In aspects, the instructions, when executed by the processor, may cause the processor to display the registered location of the endoscope on the 3D model.

In accordance with another aspect of the present disclosure, a method for performing a surgical procedure includes acquiring real-time near infrared images from a camera, retrieving a three-dimensional (3D) mode of a patient's anatomy based on pre-procedure images, displaying the 3D model, receiving an indication of a targeted critical structure within the 3D model, illuminating the patient's anatomy with near-infrared light to observe perfusion of a fluorescent dye within the patient's blood stream, identifying the targeted critical structure of the patient's anatomy within the acquired real-time near infrared images using the fluorescent dye to illuminate the targeted critical structure, registering a location of where the real-time near infrared images were acquired to the 3D model using the targeted critical structure identified in the 3D model and the acquired real-time near infrared images as a landmark, and displaying a location of where the real-time near infrared images were acquired on the 3D model.

In aspects, acquiring real-time near infrared images may include acquiring real-time near infrared images from a camera disposed within a portion of an endoscope.

In certain aspects, the method may include detecting a location, within the patient's body cavity, of an electromagnetic (EM) sensor disposed within a portion of the endoscope.

In other aspects, the method may include registering the location of the endoscope within the patient's body cavity to the 3D model using the captured real-time near infrared images.

In accordance with another aspect of the present disclosure, a method for performing a surgical procedure includes acquiring real-time images from a camera, the camera disposed within a portion of the endoscope, retrieving a three-dimensional (3D) model of a patient's anatomy based on pre-procedure images, displaying the 3D model, receiving an indication of a targeted critical structure within the 3D model, retrieving pre-procedure images including the targeted critical structure, identifying the targeted critical structure of the patient's anatomy within the retrieved pre-procedure images, comparing the acquired real-time images to the retrieved pre-procedure images, identifying the targeted critical structure within the acquired real-time images using the comparison between the acquired real-time images and the retrieved pre-procedure images, and registering the real-time images to the 3D model using the targeted critical structure identified in the 3D model and the identified targeted critical structure in the acquired real-time images as a landmark.

In aspects, the method may include detecting a location, within the patient's body cavity, of an electromagnetic (EM) sensor disposed within a portion of the endoscope.

In other aspects, the method may include registering the detected location of the endoscope to the 3D model using the targeted critical structure identified in the 3D model and the acquired real-time images as a landmark.

In certain aspects, the method may include acquiring real-time near-infrared images from a second camera disposed within a portion of the endoscope.

In aspects, the method may include displaying the acquired real-time near-infrared images.

In certain aspects, the method may include injecting a fluorescent dye into the patient's blood stream.

### BRIEF DESCRIPTION OF THE DRAWINGS

Various aspects and embodiments of the disclosure are described hereinbelow with references to the drawings, wherein:

FIG. 1 is a schematic view of an imaging system provided in accordance with the present disclosure;

FIG. 2 illustrates an endoscope inserted into a body cavity of a patient in accordance with the present disclosure;

FIG. 3 is a perspective view of a distal portion of the endoscope of FIG. 2;

FIG. 4 is a schematic view of a user interface provided in accordance with the present disclosure;

FIG. 5 is a schematic view of another user interface provided in accordance with the present disclosure;

FIG. 6 is a flow-chart depicting a method of registering a location of an endoscope relative to a generated three-dimensional model using a near infrared camera in accordance with the present disclosure.

### DETAILED DESCRIPTION

Electromagnetic tracking of medical devices within the body of a patient has increased the accuracy of representing the location of the medical device within the patient's body. The electromagnetic tracking system enables registration of pre-operative images to a real-time location of the medical device within the body of the patient to more accurately display the position of the medical device within a three-dimensional (3D) model of the patient's anatomy generated from the pre-operative images.

Fluoroscopic imaging devices have been used by clinicians, for example, to visualize the navigation of a medical device and confirm the placement of the medical device after it has been navigated to a desired location. Alternatively, near infrared (NIR) cameras can also allow the registration of real-time views from the medical device to the generated 3D model. As the medical device is navigated within the body of the patient, a dye, such as Indocyanine Green (ICG), ZW800-1, OTL38, amongst others, is injected into the patient's blood stream to illuminate critical structures within the field of view of the NIR camera. The system's software is used to isolate or segment out targeted critical structures from the generated 3D model such that the real-time images of the NIR camera and the 3D model can be registered using the targeted critical structure as the mesh point or landmark. Registration can be improved by identifying additional structures, such as the edge of the lung, bronchi, amongst others within the 3D model and the real-time images of the NIR camera.

In embodiments, the dye may be a dye having a targeted molecule that attaches to specific lesions, such as a tumor. In this manner, the dye may be injected into the patient's blood stream, after which the dye is collected or otherwise attaches to the lesions to illuminate the lesion using the NIR camera. The illuminated lesion may be used as the mesh point or landmark for registering the live images to the 3D model.

Further, the dye may be injected into the lymphatic system which then collects in the various lymph nodes thereof. The dye collected in the lymph nodes illuminates the lymph nodes in the NIR camera. As such, lymph nodes may be identified in the real-time images collected by the NIR camera, which can then be used as a mesh point or landmark to lymph nodes segmented from the 3D model to register the live images to the 3D model.

Registration can also be completed by using the software to segment out the patient's vasculature in the 3D model and then inject dye into the patient's blood stream. As the dye traverses the patient's vasculature, individual arteries and veins can be observed in the real-time images of the NIR camera, which can then be used to register the 3D model to the real-time view of the NIR camera, and therefore, the location of the medical device within the patient, using all or a portion of the vascular tree.

It is envisioned that the system may not use dye, and rather, may utilize a plurality of images of a targeted critical structure stored in a memory or network. Live images captured by a white light camera associated with an endoscope may be compared to the plurality of images of the targeted critical structure to identify the targeted critical structure in the live images. Once identified in the live images, the identified targeted critical structure may be used as a mesh point or landmark to register the 3D model to the live images.

As can be appreciated, the above processes may be automated or manually controlled. It is envisioned that a combination of automated and manual input may be utilized to improve the accuracy of the registration between the 3D model and the live images.

Although generally described with reference to the lung, it is contemplated that the systems and methods described herein may be used with any structure within the patient's body, such as the liver, kidney, prostate, gynecological, amongst others.

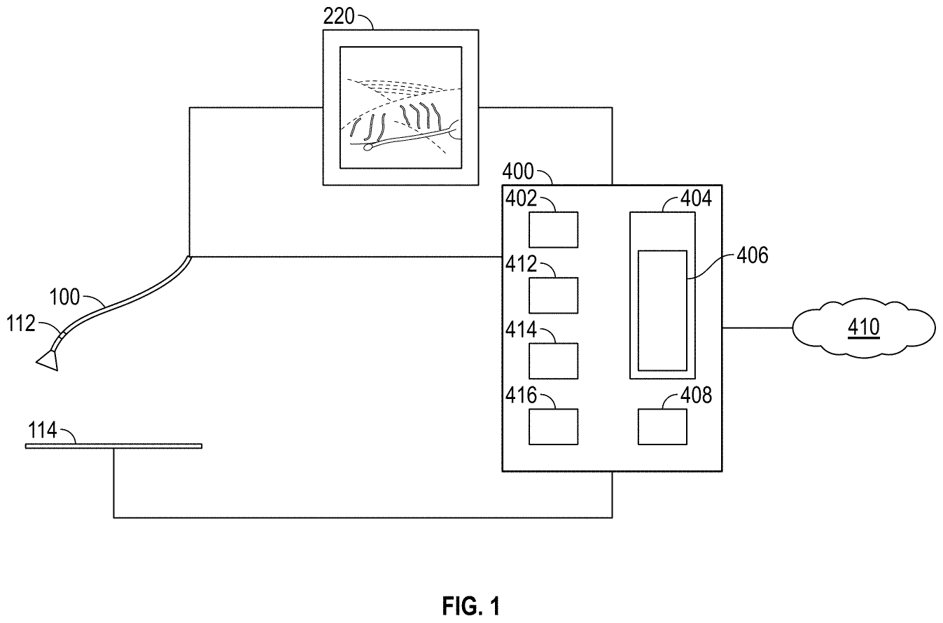

Turning now to the drawings, and with reference to FIG. 1, a system provided in accordance with the present disclosure and configured for identifying target tissue, registering real-time images captured by a surgical instrument to a generated three-dimensional (3D) model, and obtaining a tissue sample from the identified target tissue is illustrated and generally identified by reference numeral 10. The system 10 includes an endoscope 100 operably coupled to a display 220 that is configured to display one or more user interfaces 200 and 300, and which is separately connected to a workstation 400. The workstation 400 may be a desktop computer or a tower configuration with the display 220 or may be a laptop computer or other computing device. The workstation 400 includes a processor 402 which executes software stored in the memory 404. The memory 404 may store video or other imaging data captured by the endoscope 100 or pre-procedure images from, for example, a computer-tomography (CT) scan, Positron emission tomography (PET), Magnetic Resonance Imaging (MRI), Cone-beam CT, amongst others. In addition, the memory 404 may store one or more applications 406 to be executed on the processor 402. Though not explicitly illustrated, the display 220 may be incorporated into a head mounted display such as an augmented reality (AR) headset such as the HoloLens offered by Microsoft Corp.

A network interface 408 enables the workstation to communicate with a variety of other devices and systems via the Internet. The network interface 408 may connect the workstation 400 to the Internet via a wired or wireless connection. Additionally, or alternatively, the communication may be via an ad-hoc Bluetooth® or wireless networks enabling communication with a wide-area network (WAN) and/or a local area network (LAN). The network interface 408 may connect to the Internet via one or more gateways, routers, and network address translation (NAT) devices. The network interface 408 may communicate with a cloud storage system 410, in which further image data and videos may be stored. The cloud storage system 410 may be remote from or on the premises of the hospital such as in a control or hospital information technology room. An input module 412 receives inputs from an input device such as a keyboard, a mouse, voice commands, amongst others. An output device 414 connects the processor 402 and the memory 404 to a variety of output devices such as the display 220. In embodiments, the workstation may include its own display 416, which may be a touchscreen display.

In embodiments, the endoscope 100 includes a location sensor, such as an electromagnetic (EM) sensor 112 which receives electromagnetic signals from an electromagnetic field generator 114 which generates three or more electromagnetic fields. One of the applications 406 stored in the memory 404 and executed by the processor 402 may determine the position of the EM sensor 112 in the EM field generated by the electromagnetic field generator 114. The determination of the position of the endoscope 100 and one or more cameras disposed thereon enables one method in which the images captured by the endoscope may be registered to a generated 3D model of the patient's anatomy, as will be described in further detail hereinbelow. Although generally described as being an EM sensor, it is contemplated that other position sensors may be utilized, such as ultrasound sensor, flex sensors, fiber Bragg grating (FBG), robotic position detection sensors, amongst others.

In a planning or pre-procedure phase, the software stored in the memory 404 and executed by the processor 402 utilizes pre-procedure CT image data, either stored in the memory 404 or retrieved via the network interface 408, for generating and viewing a 3D model of the patient's anatomy, enabling the identification of target tissue on the 3D model (automatically, semi-automatically, or manually), and in embodiments, allowing for the selection of a pathway through the patient's anatomy to the target tissue. The 3D model may be displayed on the display 220 or another suitable display (not shown) associated with the workstation 400, or in any other suitable fashion. Using the workstation 400, various views of the 3D model may be provided and/or the 3D model may be manipulated to facilitate identification of target tissue on the 3D model and/or selection of a suitable pathway to the target tissue.

In embodiments, the software stored in the memory 404 may identify and segment out a targeted critical structure within the 3D model. It is envisioned that the segmentation may be performed automatically, manually, or a combination of both. The segmentation process isolates the targeted critical structure from the surrounding tissue in the 3D model and identifies its position within the 3D model. As can be appreciated, this position can be updated depending upon the view selected on the display 220 such that the view of the segmented targeted critical structure may approximate a view captured by the endoscope 100, as will be described in further detail hereinbelow.

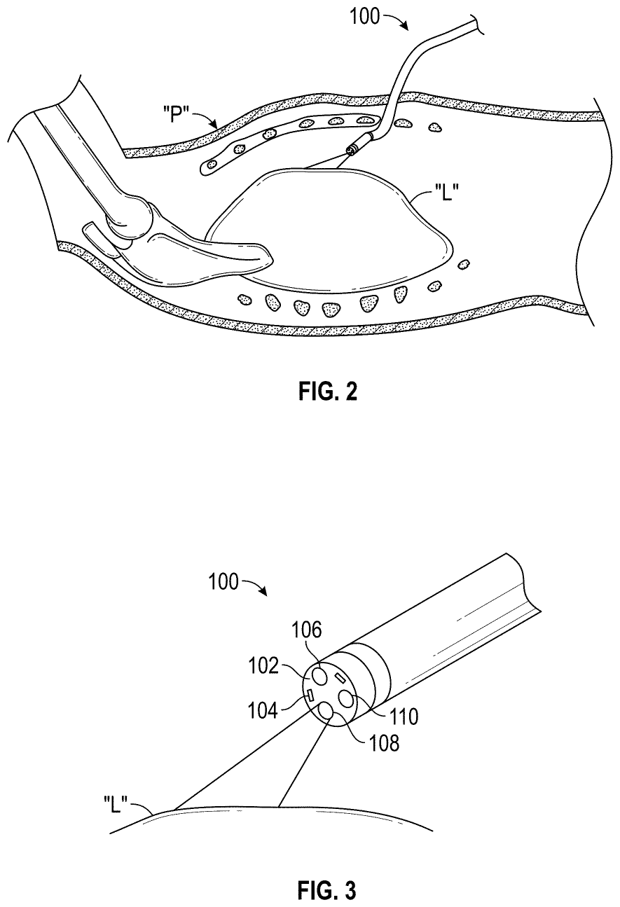

With reference to FIGS. 2 and 3, the endoscope 100 is illustrated as being inserted within the thoracic cavity of a patient “P.” As described herein, the endoscope 100 images a surface of the patient's lungs “L,” amongst other structures within the patient's thoracic cavity, as might occur during a thoracic surgery. The endoscope 100 includes a distal surface 102 having a first light source 104, a first camera 106, a second light source 108, and a second camera 110. Although generally illustrated as being disposed in a circumferential configuration, (e.g., disposed about a circumference of the distal surface 102), it is contemplated that each of the first and second light sources 104, 108 and the first and second camera 106, 110 may be disposed in any suitable configuration allowing for their serial or in-parallel use.

The first camera 106 may be a white light optical camera such as a charge-coupled device (CCD) camera, a complementary metal-oxide-semiconductor (CMOS) camera, an N-type metal-oxide-semiconductor (NMOS) camera, or any other suitable white light camera known in the art. Similarly, the first light source 104 may be or may include a light emitting diode (LED) emitting white light, although any suitable light emitting device known in the art may be utilized (e.g., the first light source 104 may be the end of an optical fiber connected to a light source that is located external to the patient). The second light source 108 may be a laser or another emitter of infrared (IR) or near infrared (NIR) light. The second camera 110 may be a CCD camera capable of detecting IR or NIR light. Other cameras capable of capturing IR or NIR light, either with or without filtering are also contemplated in connection with the present disclosure as are multi-spectral cameras such as those capable of capturing white light and NIR light using a single camera. Although generally described as having the second camera 110 disposed in the endoscope 100, it is contemplated that the second camera 110 may be disposed external to the patient. In this manner, the second camera 110 may be disposed on a boom or arm (not shown) that is operably coupled to the workstation 400

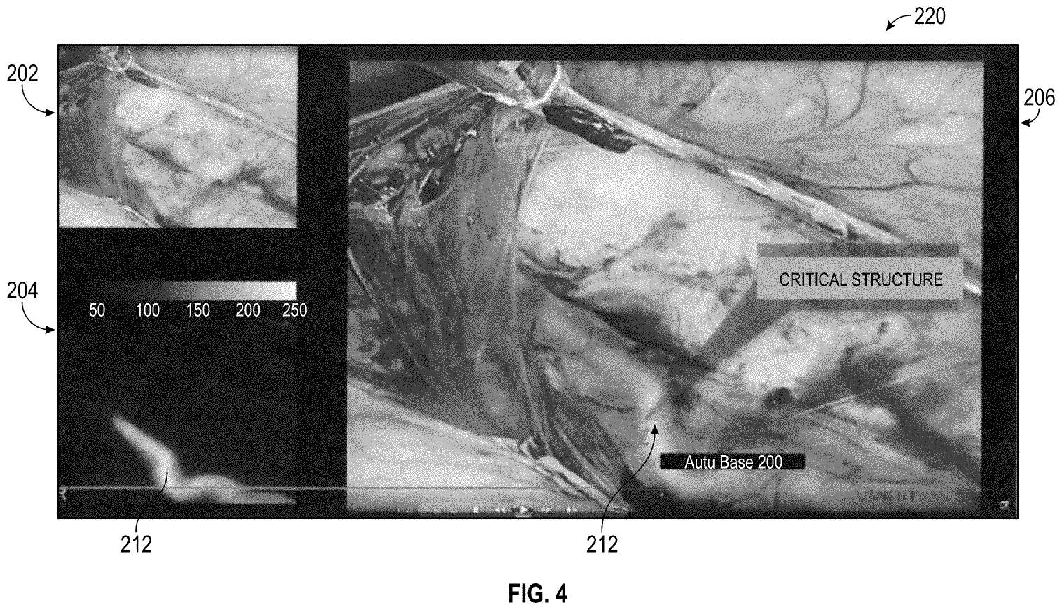

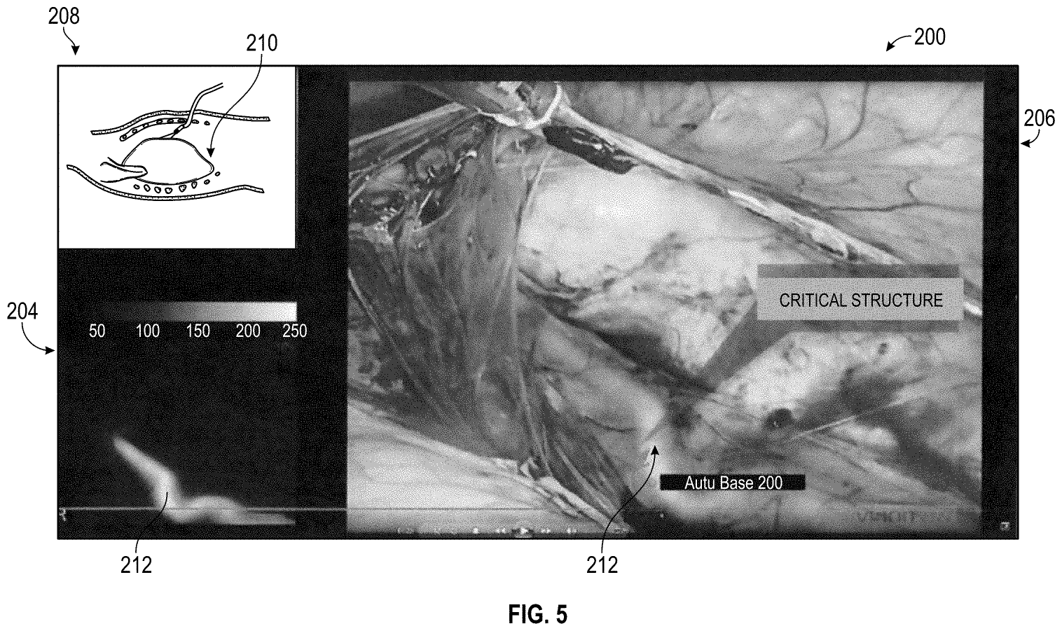

During a procedure, following insertion, as illustrated in FIG. 2, the first light source 104 and the first camera 106 are used for general navigation within the body of the patient. During this time, in addition to images captured by the combination of the first light source 104 and the first camera 106, the position of the EM sensor 112 (FIG. 2) in the EM field generated by the electromagnetic field generator 114 is monitored and displayed within the 3D model of the patient's anatomy on the display 220. The white light emitted by the first light source 104 reflects off one or more tissues of the body and is captured by the first camera 106. The images from the first camera 106 are displayed on a display, as illustrated in FIG. 4. Additionally, FIG. 4 illustrates a user interface 200 depicting an image 202 captured by the first camera 106, an image 204 captured by the second camera 110, and a composite image 206 illustrating the image 202 captured by the second camera 110 overlayed on the image 204 captured by the first camera 106. In embodiments, it is contemplated that the user interface 200 may display only the images captured by the first camera 106, only the images captured by the second camera 110, only the combined images captured by the first and second cameras 106, 110, or combinations thereof. In one non-limiting embodiment, one or more of the images displayed may be replaced by a representation 208 of the generated 3D model of the patient's anatomy (FIG. 5) and the position of the endoscope 100 may be depicted on the 3D model using a marker 210 or any other suitable method of indicating the position and/or orientation of the endoscope 100 within the patient's anatomy. In embodiments, the 3D model may be displayed on a separate display (not shown) associated with the workstation 400.

During navigation of the endoscope 100 through the thoracic cavity, the position of the endoscope 100 may be detected using the EM sensor 112 and the EM field generator 114. The detected position of the endoscope 100 and white light images captured by the first camera 106 are utilized to initially register the real-time position of the endoscope within the patient's anatomy and to the generated 3D model of the patient's anatomy. Although this method of registration is generally accurate, the accuracy can be further improved by identifying critical structures within the images captured by the endoscope 100. Such an improvement in accuracy is particularly useful when the endoscope is adjacent the target tissue and the treatment or taking a biopsy of the correct tissue is of increased importance.

In this manner, various dyes, such as ICG, ZW800-1, OTL38, amongst others, may be injected into the blood stream of the patient. When the ICG dye is illuminated with certain frequencies of light in the IR or NIR range, the ICG fluoresces an infrared color that can be captured by a camera such as the second camera 110. Capturing the NIR images can be triggered automatically or as directed by the clinician. The clinician can instruct or control the system to capture the endoscopic image using any suitable method of control available (e.g., a footswitch, by voice, an assistant, a hand motion, amongst others). Alternatively, the system can perform this task automatically by detecting sudden changes in the image.

When dyes such as ICG, which generate visible changes, are used the results are seen in the user interface 200. The process of the dye diffusing through the tissue reveals anatomic detail, such as vasculature and parts of the lymphatic system. As can be appreciated, as the blood having the ICG dye is transported through the blood vessels, the blood vessels will, for a time, appear distinct from the surrounding tissue when illuminated with an NIR light source. Thus, by illuminating the organ tissues with NIR light from the second light source 108, as illustrated in image 204 of the user interface 300 (FIG. 4) and capturing the returned fluorescence from the ICG in the blood stream using the second camera 110, as illustrated in image 206 of the user interface 300, blood vessels will appear in the user interface 300 as being illuminated using a color in the visible light range, such as green, as shown in FIGS. 4 and 5. In this manner, a targeted critical structure identified within the 3D model can be identified within the images captured by the first and second cameras 106, 110 and a marker or other method of depicting the location of the illuminated targeted critical structure 212 within the image 204 captured by the second camera 110 and the composite image 206.

Although generally described as being injected into the blood stream of the patient. It is contemplated that the dye may be injected into a lymph node, target tissue (e.g., tumor or the like), amongst others. In this manner, the dye flows throughout the lymphatic system and is collected by the various lymph nodes of the patient's body. As can be appreciated, the dye collected in the lymph nodes enables each lymph node of the lymphatic system to be identified using NIR light captured by the second camera 110. The identified lymph nodes may then be utilized as a mesh point or landmark to register the 3D model to the real time image captured by the second camera 110.

In embodiments, the dye may be a targeted molecule, such as OTL38, that attaches to tumors or other similar lesions. In this manner, after the dye is injected into the blood stream, the dye collects or otherwise attaches to the tumor to illuminate the tumor in the live images captured by the second camera 110. Using this approach, it is envisioned that where two lesions are identified in the CT images, and only one lesion can be identified using the NIR light captured by the second camera 110, the location of the illuminated first lesion can be utilized to identify the location of the second tumor in the live images captured by the second camera 110, and therefore, the 3D model.

As can be appreciated, utilizing dyes that target or otherwise attach to specific structures within the patient's body may reduce the amount of time it takes to identify the target critical structure within the images captured by the second camera 110. In this manner, a dye that attaches to a specific type of lesion, such as a tumor, may be injected and then the patient's anatomy may be monitored until the targeted structure is illuminated by the dye in the images captured by the second camera. Using the targeted structure as the mesh point or landmark in both the 3D model and the images captured by the second camera 110 enables accurate registration between the 3D model and the real-time images captured by the second camera.

In one non-limiting embodiment, using the workstation 400, the pulmonary artery (or more specifically, the left-pulmonary artery) may be segmented from or isolated within the 3D model of the patient's lungs. When the endoscope 100 has been navigated to a position within the patient's thoracic cavity where the left-pulmonary artery is believed to be located, the ICG dye is injected into the patient's blood stream, which then diffuses through critical structures within the patient's lungs, causing these critical structures, including the left-pulmonary artery, to illuminate when viewed by the second camera 110. Illumination of the left-pulmonary artery is then identified within the images captured by the second camera 110 by the workstation 400, which then correlates or otherwise registers the real-time location of the left-pulmonary artery within the images captured by the second camera 110 with the 3D model using the left-pulmonary artery as the mesh point or landmark to more accurately reflect the position of the endoscope 100 within the 3D model illustrated on the display 220.

It is envisioned that the workstation 400 may match or otherwise register the NIR images captured by the second camera 110 to the 3D model automatically, such as by least squares fitting or other suitable means, or by manually aligning the images on the workstation 400. In embodiments, combinations of automated and manual input may be utilized.

In embodiments, the time at which structures are illuminated by the dye may be utilized to help identify critical structures within the patient's anatomy. For example, as the dye flows throughout the vasculature, arteries fill with the dye first, followed by smaller arteries and eventually veins. It is contemplated that the timing of when these vascular structures are illuminated may be utilized to improve segmentation of the structures in the 3D model.

As can be appreciated, adding additional structures to the registration process can improve registration between the real-time images captured by the endoscope 100 and the 3D model. In this manner, structures like the edge of the patient's lung, bronchi, bile ducts, nerves, amongst others, can be segmented out or otherwise isolated along with the left-pulmonary artery to improve registration using ICG dye and the second camera 110. Furthermore, it is contemplated that the workstation 400 may segment out or otherwise isolate all of the vasculature of the patient's lungs in the 3D model such that as the ICG dye diffuses within the vasculature tree in real time, the workstation 400 can identify arteries and veins within the patient's anatomy and use their location within the real-time images captured by the second camera 110 to register the real-time images, and therefore the location of the endoscope, to the 3D model. In this manner, multiple structures identified in the real-time images captured by the second camera 110, and multiple structures in the 3D model corresponding to the multiple identified structures in the real time images may be utilized to improve the accuracy of the registration of the 3D model to the real-time images captured by the endoscope 100.

In embodiments, the position of the endoscope 100 within the patient's body cavity may be monitored and identified based solely upon the images captured by the first camera 106 and the second camera 110. In this manner, as the endoscope 100 is navigated within the patient's body cavity, the ICG dye is injected into the blood stream and the illuminated targeted critical structure is observed by the second camera 110, as described in detail hereinabove. The illuminated targeted critical structure is correlated or registered a corresponding targeted critical structure within the 3D model, at which point the location at which the images captured by the first and second camera 106, 108, and therefore, the location of the endoscope 100 within the patient's body cavity can be identified and displayed on the 3D model. It is envisioned that the EM sensor 112 and the second camera 110 can be used at different times, concurrently, etc. depending upon the needs of the surgical procedure and the level of accuracy that is needed.

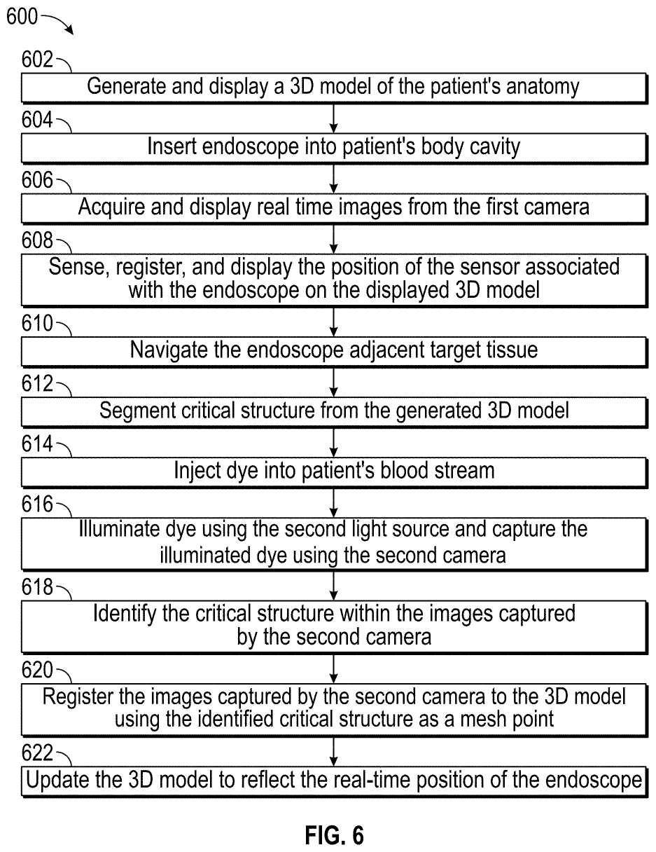

With reference to FIG. 6, a method of utilizing dye diffusion and NIR light to register real-time images of a patient's anatomy to a generated 3D model is illustrated. Initially, in step 602, the workstation 400 generates a 3D model of the patient's anatomy, including the patient's lungs, using pre-procedure CT images or the like (e.g., MRI, etc.), which is then displayed on the display 220 associated within the workstation 400. Thereafter, in step 604, the endoscope 100 is inserted into the patient's thoracic cavity. In step 606, real-time images of the patient's anatomy are captured by the first camera 106 and displayed on the display 220. In step 608, the position of the sensor 112 associated with the endoscope 100 is sensed by the EM field generator 114 and the software associated with the workstation 400 registers the location of the sensor 112 to the generated 3D model and depicts the real-time location of the endoscope 100 within the patient's anatomy on the display 220 to aid the clinician in identifying the position of the endoscope 100 relative to targeted critical structures and target tissue. The endoscope 100 is navigated to a position adjacent the target tissue in step 610 and the workstation 400 segments or otherwise isolates critical structures, such as the left-pulmonary artery, in the 3D model in step 612. In step 614, ICG dye or any other suitable dye is injected into the patient's blood stream and diffuses within structures of the patient's anatomy. The ICG dye is illuminated using NIR light and captured by the second camera 110 in step 616 to identify critical structures within the patient's anatomy. In step 618, the targeted critical structure identified in step 612 is identified in the real-time images captured by the second camera 110 using fluorescence, and in step 620, the identified targeted critical structure in the captured real-time images is registered to the 3D model using the identified targeted critical structure as the mesh point or landmark. Once registered, the 3D model is updated in step 622 to accurately reflect the real-time position of the endoscope 100 within the 3D model and with respect to the target tissue, such that the target tissue may be treated accurately and/or a biopsy may be obtained more accurately.

Alternatively, it is envisioned that the ICG dye may be injected into a lymph node, target tissue (e.g., tumor or the like), amongst others. In this manner, the dye flows throughout the lymphatic system and is collected by the various lymph nodes of the patient's body. As can be appreciated, the dye collected in the lymph nodes enables each lymph node of the lymphatic system to be identified using NIR light captured by the second camera 110. The identified lymph nodes may then be utilized as a mesh point or landmark to register the 3D model to the real time image captured by the second camera 110.

Although generally described as using dye to illuminate critical structures within the patient's anatomy, it is contemplated that the system 10 may forego the use of dyes and identify critical structures within the white light images captured by the first camera 106 by comparing the images captured by the first camera 106 to a plurality of images of critical structures either stored in the memory 404 or retrieved via the network interface 408. In this manner, the system 10 can automatically compare the images captured by the first camera 106 to the stored images and identify matches or similar landmarks within the two sets of images. In embodiments, the system 10 identify images including a pre-identified critical structure, such as the left-pulmonary artery, and compare the images including the pre-identified critical structure to the live images captured by the first camera 106. When a match is identified, the system 10 may use the identified critical structure in the live images captured by the first camera 106 as a mesh point or landmark to register the live images captured by the first camera 106 to the 3D model.

In embodiments, the system 10 may compile a plurality of images including the pre-identified critical structure to which the live images captured by the first camera 106 may be compared. As can be appreciated, utilizing multiple images including the pre-identified critical structure may increase the accuracy of the system 10 identifying the critical structure within the live images. Although generally described as being an automated process, it is envisioned that the system 10 may receive user input to identify the critical structure within the live images or to confirm automated identification of the critical structure within the live images, or combinations of automated identification and manual inputs.

Although described generally hereinabove, it is envisioned that the memory 404 may include any non-transitory computer-readable storage media for storing data and/or software including instructions that are executable by the processor 402 and which control the operation of workstation 400 and, in some embodiments, may also control the operation of the endoscope 100. In an embodiment, memory 404 may include one or more storage devices such as solid-state storage devices, e.g., flash memory chips. Alternatively, or in addition to the one or more solid-state storage devices, the memory 404 may include one or more mass storage devices connected to the processor 402 through a mass storage controller (not shown) and a communications bus (not shown).

Although the description of computer-readable media contained herein refers to solid-state storage, it should be appreciated by those skilled in the art that computer-readable storage media can be any available media that can be accessed by the processor 406. That is, computer readable storage media may include non-transitory, volatile and non-volatile, removable and non-removable media implemented in any method or technology for storage of information such as computer-readable instructions, data structures, program modules or other data. For example, computer-readable storage media may include RAM, ROM, EPROM, EEPROM, flash memory or other solid-state memory technology, CD-ROM, DVD, Blu-Ray or other optical storage, magnetic cassettes, magnetic tape, magnetic disk storage or other magnetic storage devices, or any other medium which may be used to store the desired information, and which may be accessed by the workstation 400.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of embodiments. Those skilled in the art will envision other modifications within the scope and spirit of the claims appended hereto.

What is claimed is:

1. A system for performing a surgical procedure, comprising:
a camera configured to capture real-time near infrared images;
an injection system configured to inject a fluorescent dye into a patient's blood stream; and
a workstation operably coupled to the camera, the workstation including a memory and a processor, the memory storing instructions, which when executed by the processor cause the processor to:
retrieve a three-dimensional (3D) model of the patient's anatomy based on pre-procedure images stored on the memory;
receive an indication of a targeted critical structure within the 3D model;
observe, using the captured real-time near infrared images, perfusion of the fluorescent dye through tissue to identify critical structures illuminated by near-infrared light; and
register the real-time near-infrared images to the 3D model using the identified illuminated critical structure in the real-time near infrared images captured by the camera and the identified targeted critical structure in the 3D model as a landmark.

2. The system according to claim 1, further including a second camera configured to capture real-time white light images.

3. The system according to claim 1, further including an endoscope, wherein the camera is disposed within a portion of the endoscope.

4. The system according to claim 2, further including an endoscope, wherein the camera and the second camera are disposed within a portion of the endoscope.

5. The system according to claim 1, wherein the fluorescent dye is Indocyanine green dye.

6. The system according to claim 1, wherein the instructions, when executed by the processor, cause the processor to identify and segment a targeted critical structure from the 3D model.

7. The system according to claim 1, wherein the instructions, when executed by the processor, further cause the processor to display a location where the real-time near infrared images were captured on the registered 3D model.

8. The system according to claim 3, further including an electromagnetic (EM) sensor disposed within a portion of the endoscope, wherein the workstation is configured to sense the location of the EM sensor within the patient's body cavity.

9. The system according to claim 8, wherein the instructions, when executed by the processor, cause the processor to register a location of the endoscope within the body cavity of the patient to the 3D model of the patient's anatomy using the captured real-time near infrared images.

10. The system according to claim 9, wherein the instructions, when executed by the processor, cause the processor to display the registered location of the endoscope on the 3D model.

11. A method for performing a surgical procedure, comprising:
acquiring real-time near infrared images from a camera;
retrieving a three-dimensional (3D) model of a patient's anatomy based on pre-procedure images;
displaying the 3D model;
receiving an indication of a targeted critical structure within the 3D model;
illuminating the patient's anatomy with near infrared light to observe perfusion of a fluorescent dye within the patient's blood stream;
identifying the targeted critical structure of the patient's anatomy within the acquired real-time near infrared images using the fluorescent dye to illuminate the targeted critical structure;
registering a location of where the real-time near infrared images were acquired to the 3D model using the targeted critical structure identified in the 3D model and the acquired real-time near infrared images as a landmark; and
displaying a location of where the real-time near infrared images were acquired on the 3D model.

12. The method according to claim 11, wherein acquiring real-time near infrared images includes acquiring real-time near infrared images from a camera disposed within a portion of an endoscope.

13. The method according to claim 12, further comprising detecting a location, within the patient's body cavity, of an electromagnetic (EM) sensor disposed within a portion of the endoscope.

14. The method according to claim 13, further comprising registering the location of the endoscope within the patient's body cavity to the 3D model using the captured real-time near infrared images.

15. A method for performing a surgical procedure, comprising:
acquiring real-time images from a camera, the camera disposed within a portion of an endoscope;
retrieving a three-dimensional (3D) model of a patient's anatomy based on pre-procedure images;
displaying the 3D model;
receiving an indication of a targeted critical structure within the 3D model;
retrieving pre-procedure images including the targeted critical structure;
identifying the targeted critical structure of the patient's anatomy within the retrieved pre-procedure images;
comparing the acquired real-time images to the retrieved pre-procedure images;
identifying the targeted critical structure within the acquired real-time images using the comparison between the acquired real-time images and the retrieved pre-procedure images; and
registering the real-time images to the 3D model using the targeted critical structure identified in the 3D model and the identified targeted critical structure in the acquired real-time images as a landmark.

16. The method according to claim 15, further comprising detecting a location, within the patient's body cavity, of an electromagnetic (EM) sensor disposed within a portion of the endoscope.

17. The method according to claim 16, further comprising registering the detected location of the endoscope to the 3D model using the targeted critical structure identified in the 3D model and the acquired real-time images as a landmark.

18. The method according to claim 15, further comprising acquiring real-time near-infrared images from a second camera disposed within a portion of the endoscope.

19. The method according to claim 18, further comprising displaying the acquired real-time near-infrared images.

20. The method according to claim 15, further comprising injecting a fluorescent dye into the patient's blood stream.
